(19) United States
(12) Patent Application Publication
(10) Pub. No.: US 2007/0239026 A1
(43) Pub. Date: Oct. 11, 2007

(54) CONTROLLING A MEDICAL NAVIGATION SOFTWARE USING A CLICK SIGNAL

(75) Inventors: Martin Immerz, Grafelfing (DE); Wolfgang Steinle, Munich (DE)

Correspondence Address: DON W. BULSON (BrainLAB), RENNER, OTTO, BOISSELLE & SKLAR, LLP, 1621 EUCLID AVENUE - 19TH FLOOR, CLEVELAND, OH, 44115, US

(73) Assignee: BRAINLAB AG, Feldkirchen (DE)

(21) Appl. No.: 11/562,740

(22) Filed: Nov. 22, 2006

Related U.S. Application Data
(60) Provisional application No. 60/754,507, filed on Dec. 28, 2005.

(30) Foreign Application Priority Data
Nov. 24, 2005 (EP) 05 025 655.1-2318

(51) Int. Cl. A61B 8/14

(52) U.S. Cl. 600/463

(57) ABSTRACT

A method for controlling a computer-assisted, medical navigation system that provides assistance for a particular part of a treatment, includes using an acoustic mechanism to generate a click signal; and the medical navigation system providing treatment assistance based on the click signal.

### RELATED APPLICATION DATA

This application claims priority of U.S. Provisional Application No. 60/754,507 filed on Dec. 28, 2005, which is incorporated herein by reference in its entirety.

### FIELD OF THE INVENTION

The invention relates to controlling medical navigation software and, more particularly, to a method for commanding a medical navigation via an acoustic signal and to a device that can produce the acoustic signal.

### BACKGROUND OF THE INVENTION

In computer-assisted surgery, the surgeon typically is provided with different image outputs on a display screen. In most cases, the image outputs are provided by a computer-assisted, medical navigation system located within an operating theater. These image outputs typically show under the control of a software control system at least parts of patient data sets such as, for example, three-dimensional or sectional representations of parts of the patient's body. The patient data sets can be generated either by imaging methods such as, for example, CT or MR tomography, x-ray, ultrasound or fluoroscopy, or by image-free methods such as tapping the surface of a bone with a registered pointing instrument or by laser scanning. Within the scope of image assistance, instruments or treatment means also can be shown in their positional relationship to the patient data by means of a tracking system included in the navigation system, so as to ensure that the surgeon is assisted.

Depending on the progress of treatment, it is often necessary to display a specific part of the software assistance on the image output, namely the part including the functions utilized for the current treatment step. The software also may be said to include different “pages” that interchange in the course of the treatment.

In image-assisted applications in the hip or knee region, for example, particular software pages typically are selected during treatment. Conventionally, this is achieved by means of a touch-sensitive screen (touch screen), wherein the corresponding inputs are entered by touching “cells” on the screen. Other types of software control also are known, including, for example, using a foot switch, a virtual keyboard, voice control or navigated and/or operable instruments that perform a particular action that initiates an action within the software control system.

The act of switching or paging through screens can be very distracting to the surgeon, particularly, when such actions interrupt the medical procedure. The acceptance of image assistance, which is otherwise regarded as highly advantageous, suffers from this.

Acoustic control using spoken commands has also proven unsatisfactory for controlling software applications. So-called “voice-controlled” control systems often meet with resistance from surgeons, since many people simply find it difficult to talk to a computer, particularly when others are present. Moreover, the medical team normally speak with each other during a procedure, and there exists the possibility that the control system may respond to someone other than the surgeon. Also, in almost all cases, the surgeon wears a face mask, a splash guard and/or other facial protection. This facial protection can distort spoken acoustic signals and render them unrecognisable to the system.

### SUMMARY OF THE INVENTION

The present invention provides a system, device and method for controlling a computer-assisted, medical navigation system, wherein a non-verbal acoustic signal, such as a click signal, is used to initiate an action in the navigation system and thus command the system to perform a certain action. The acoustic signal is provided to the navigation system, which then processes the signal so as to perform a particular command. The acoustic signal is generated by a manually operated device, such as a clicker, that may be mounted to or integrated in a surgical instrument, for example. The system includes a receiver for detecting the non-verbal acoustic signal and means responsive to the received signal for initiating a command function. The acoustic signal although non-verbal, preferably is audible to provide immediate feedback to the surgeon or other medical personnel that the signal has been generated.

The acoustic signal, which is clearly distinguishable and identifiable, can trigger a particular action (e.g., in the image output). An acoustic signal such as a click or chirp signal is simple and short and can be easily provided at a volume above a “room volume”, i.e., above the normal volume during a medical treatment. A click signal is distinct, clear, unambiguous, audible and distinguishable from other acoustic signals in an operating theater. In particular, a click signal is easy to create, and can be provided by a “clicker”, e.g. an object that abruptly changes shape when manipulated. In principle, however, the acoustic signal also can be generated by an impact and/or by one object hitting another object, as well as by other means.

The control realized is very stable, particularly when compared to voice control. The unambiguously distinguishable acoustic signal can be easily detected and filtered out. Also, such a click signal is likely to be more accepted than a voice recognition computer. It is unlikely that an acoustic signal, such as a click signal, will be disrupted by other sounds. An acoustic signal embodied as a click signal has a unique frequency, wherein it can be formed to be very stable and is reliable. Further, a device that creates such signal can be simple and, thus, easy to operate, clean, autoclave or sterilize. Additionally, the device that creates the acoustic signal can be configured to operate without electrical power and, therefore, does not need an external power connection or internal batteries to operate.

A suitable part of a computer-assisted medical navigation system, wherein different image outputs are provided within the scope of navigation, can be controlled via an acoustic signal, such as a click signal. The click signal can be generated by an acoustic device, and the signal can be provided to the navigation system, whereupon the navigation system executes a particular command or commands when provided with the signal (e.g., change the image output). In a preferred embodiment, different image outputs are provided within the scope of navigation, and when the navigation system receives the acoustic signal, the image output is changed accordingly.

Switching the image output over, forward or backward, can be triggered by the acoustic signal. The acoustic signal, for example, can be generated by a separate click mechanism or by a click mechanism that is arranged on a medical instrument.

There exists the option of generating two or more acoustic signals that differ in tone, in particular in pitch. Additionally or in combination, successive acoustic signals can be generated in different rhythms, e.g., two clicks in quick or slow succession. Also, the duration of the acoustic signal may be varied or other wised controlled to signify different commands (preferably the duration of the acoustic signal is less than one second). This provides different and versatile options for actuating different actions, such as for paging forward or backward through the image outputs within an output sequence.

A system for controlling a medical navigation system includes such a medical navigation and/or image-assisting system and a controller for the system responsive to an input. Further, the system includes an acoustic generation device and an acoustic receiving device assigned to the navigation system.

The controller can be integrated in the navigation system and can include a switching unit for switching the image output over, forward or backward in response to the acoustic signal. The device can include a separate acoustic mechanism or the mechanism can be arranged on a medical instrument, in particular arranged such that it can be removed and/or replaced.

The acoustic mechanism can include a deformable planar structure, preferably made of metal. This planar structure can experience an abrupt change in shape by mechanical deformation and can thus emit the acoustic signal. In general terms, one possible type of function for the acoustic mechanism is that an abrupt change in the shape of a body causes the signal to be generated. Good acoustic signals, such as click signals, can be obtained when a pre-shaped portion is abruptly deformed, e.g., a dent or indentation in a metal strip. An easily distinguishable acoustic signal is in particular generated when said metal body is manipulated such that the dent inverts towards the other planar side (and possibly also back into its initial position again).

The acoustic mechanism can generate two or more acoustic signals that differ in tone, in particular in pitch, particularly by changing the shape of different portions or of the same portion in different directions. Further, two or more acoustic mechanisms can be provided in order to generate different acoustic signals.

The navigation system includes an image output unit, and the controller controls the display on the image output unit, wherein an image output is manipulated, such as, for example, paging forward or backward through pages of the image output within the framework of a software workflow.

The system and method provides a suitable part of a medical navigation system and/or a suitable image output of a medical navigation system and includes a medical navigation and/or image assisting system having an image output unit, and a control scheme or controller for displaying on the image output unit in response to an input.

### BRIEF DESCRIPTION OF THE DRAWINGS

The forgoing and other features of the invention are hereinafter discussed with reference to the drawings.

FIG. 1 illustrates an exemplary system in accordance with the invention for controlling a medical navigation system using an acoustic signal.

### DETAILED DESCRIPTION

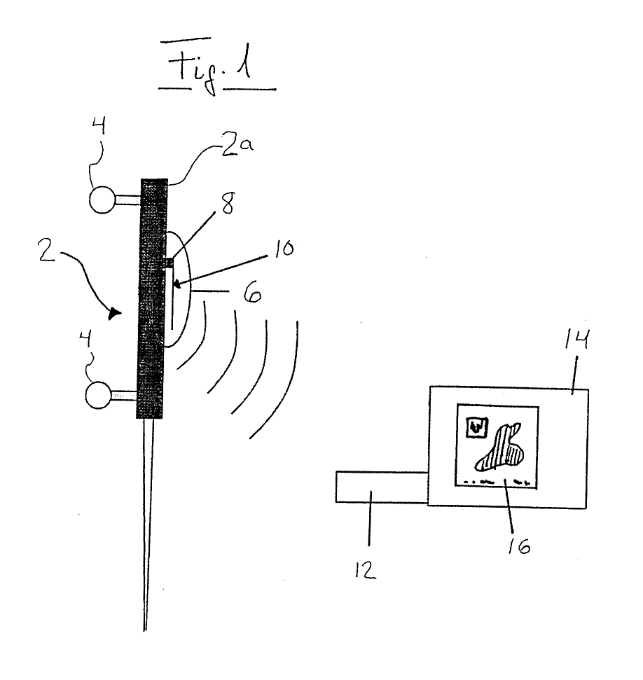

Referring to FIG. 1, a surgical instrument 2, such as a probe, for example, includes navigation reflector markers 4. An acoustic mechanism 6, such as a click mechanism 6, is coupled to a handle 2a of the probe 2 and can be sub-divided into a mount 8 and a small metal plate 10. Arranged on the medical instrument 2, the acoustic mechanism 6 can be easily accessed by the surgeon. The mechanism 6 need not be arranged laterally on the handle 2a , as shown in FIG. 1, but could equally be provided on the rear or top portion of the handle 2a, as a button switch or the like. This location can be advantageous as it may be less of a disturbance when holding the instrument 2.

In the embodiment shown in FIG. 1, the acoustic mechanism 6 includes the small metal plate 10, which emits a distinguishable acoustic signal when it is deformed, such as a click signal, for example. When this distinguishable click sound is detected by a microphone 12 coupled to a navigation system 14, predefined software steps can be activated. In most cases, the application will be one which allows stepping through a workflow that includes different software screen pages. FIG. 1, for example, shows screen 16 that shows a display currently assisting the work of the surgeon. If a particular action is finished, then generating a click signal using the acoustic mechanism 6 can call up a subsequent screen page that explicitly assists the next action by the surgeon.

In principle, it is possible to trigger any actions via the acoustic signal in order to assist the treatment, and other possible actions, for example, could be importing image data sets into the navigation system, beginning a registration or re-registration for parts of the patient's body or instruments, and launching a treatment-assisting apparatus.

Although not shown in the drawing, it is also conceivable, for example, to use two different small plates that emit different acoustic signals when they are deformed, such that different functionalities can be triggered or enabled, such as for example “switching forward” or “switching backward” within a particular sequence. Such embodiments in which a number of different click signals are provided can of course also be constructed differently, for example as a rocker switch.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A method for controlling a computer-assisted, medical navigation system, comprising the step of using a manually operated acoustic mechanism to generate a non-verbal acoustic signal for initiating a action in the navigation system.

2. The method according to claim 1, where in the device generates a click signal.

3. The method according to claim 2, wherein the acoustic mechanism is provided on a medical instrument.

4. The method according to claim 2, wherein using step includes generating at least two click signals that differ in tone, pitch and/or rhythm.

5. The method according to claim 1, wherein the action initiated by the navigation system includes providing a different image output on a display of the medical navigation system.

6. The method of claim 1, wherein the non-verbal acoustic signal is an audible signal having a duration no greater than one second.

7. A command system for controlling a medical navigation system, comprising an acoustic receiving device for receiving a non-verbal acoustic signal, and a processor for processing the acoustic signal and initiating a command to the navigation system.

8. The command system according to claim 7, further comprising an acoustic generation device for generating the non-verbal acoustic signal.

9. The command system according to claim 7, in combination with the medical navigation system, wherein the device is integrated into the system.

10. The command system according to claim 7, wherein the controller is integrated in the navigation system.

11. The command system according to claim 7, wherein the acoustic generation device include an attachment mechanism for attachment to a medical instrument.

12. The command system according to claim 7, further comprising a medical instrument, wherein said acoustic generation device is provided on the medical instrument.

13. The device according to claim 7, wherein the acoustic generation device includes a clicker.

14. The device according to claim 7, wherein the acoustic generation device is operable to generate at least two click signals that differ in tone and/or pitch.

14. The device according to claim 13, wherein the at least two click signals are generated by a change in shape of the acoustic generation device.

15. The device according to claim 7, wherein the acoustic generation device comprises at least two acoustic generation devices, each of the at least two acoustic generation devices operative to generate different click signals.

16. The device according to claim 7, wherein the navigation system includes an image output unit, and the controller controls the display on the image output unit.

17. The device according to claim 16, wherein the controller is operative to change the image output so as to page forward or backward in an image output sequence of a software workflow.
